(19) United States
(12) Patent Application Publication
(10) Pub. No.: US 2012/0244616 A1
(43) Pub. Date: Sep. 27, 2012

(54) METHOD OF OOCYTE CRYOPRESERVATION USING ANTIFREEZE PROTEIN

(75) Inventors: Jun Woo Jo, Seoul (KR); Chang Suk Suh, Seoul (KR); Byung Chul Jee, Seoul (KR)

(73) Assignee: SEOUL NATIONAL UNIVERSITY BUNDANG HOSPITAL, SEONGNAM (KR)

(21) Appl. No.: 13/412,293

(22) Filed: Mar. 5, 2012

(30) Foreign Application Priority Data
Mar. 4, 2011 (KR) 10-2011-0019521

(51) Int. Cl. C12N 5/075

(52) U.S. Cl. 435/366

(57) ABSTRACT

Disclosed is a method for cryopreserving an oocyte by adding an antifreeze protein to a cryopreservation liquid (equilibrium solution, vitrification solution). The disclosed cryopreservation method of an oocyte minimizes damage to the oocyte, which increases the survival rate of the oocyte after freezing and thawing of the oocyte, and improves a fertilization rate, and a blastocyst development ratio of the oocyte.

### CROSS-REFERENCES TO RELATED APPLICATIONS

This application is a U.S. Utility application, which claims priority to Korean Application No. 10-2011-0019521, filed Mar. 4, 2011. The entire contents of the aforementioned patent application are incorporated herein by this reference.

### TECHNICAL FIELD

The present invention relates to a method for cryopreserving an oocyte by adding an antifreeze protein to each cryopreservation liquid.

### BACKGROUND ART

Oocyte cryopreservation is a very attractive method capable of preserving fertility in the case where a large amount of oocytes are obtained during in vitro fertilization, in the case where semen collection is failed on the day of in vitro fertilization, in the case where fertility of a cancer patient is required to be preserved, or in the case where there is a concern about loss of fertility due to endometriosis, etc. An oocyte freezing method includes a slow freezing method and a vitrification method.

The cryopreservation compound and the additive include a mixed solution containing a permeable cryopreservation compound (Ali and Sheton, 1993; Kasai, 1997; Wright et al., 2004) such as Propanediol (PROH), glycol (glycerol), ethylene glycol(EG), dimethyl-sulfoxide (DMSO), and a non-permeable cryopreservation compound (Leibo and Oda, 1992; Naitana et al., 1997; Ohboshi et al., 1997; Shaw et al., 1997; Kuleshova et al., 2001; Asada et al., 2002) such as sucrose, threhalose, Ficoll, dextran, polyvinyl pyrrolidone, polyethylene glycol, wherein a mixture of the permeable cryopreservation compound and the non-permeable cryopreservation compound is used in a freezing adjuvant of a hyperacute vitrification method. Among of them, EG is the most frequently used freezing adjuvant due to its low molecular weight and low toxicity (Chian et al., 2004). Meanwhile, there is a report that the combination of two or more freezing adjuvants is more effective (Vajta et al., 1998).

An embryo is generally frozen and thawed in the use of infertility treatment. Meanwhile, an oocyte has a large size and accordingly, a large amount of moisture, and thus a frozen oocyte may be damaged. Accordingly, it is reported that freezing treatment of oocyte has a low success rate. Also, a matured oocyte may be subjected to a damage causing deformation of a meiosis spindle fiber, which reduces cleavage capability or blastocyst development ratio (Carrol et al., 1993; Lane et al., 1999; Hong et al., 1999, Shaw et al., 2000;). As described above, in spite of much research on a freezing method, a significant progress in improvement for minimizing the freezing damage of an oocyte has not been achieved.

Accordingly, the inventors of the present invention found a method for minimizing damage to a spindle fiber, damage to an oocyte membrane, and damage to a fine cell structure within an oocyte during freezing of the oocyte, which increases the survival rate of the oocyte after thawing of the oocyte, and improves a fertilization rate, and a blastocyst development ratio of the oocyte. Then, based on this finding, they completed this invention.

### SUMMARY OF THE DISCLOSURE

The present invention relates to a method for minimizing damage to an oocyte during freezing of the oocyte by using a cryopreservation liquid added with an antifreeze protein, which increases a survival rate, a fertilization rate and a blastocyst development ratio of the oocyte.

To accomplish the above objectives of the present invention, it provides a method for cryopreserving a cell, the method comprising the steps of: preparing a composition for cryopreservation by mixing 5 to 50 vol % of serum, 10 to 20 vol % of ethylene glycol (EG), 10 to 20 vol % of propylene glycol and 100 to 3000 ng/ml of antifreeze protein; and suspending and freezing the cell with the composition.

In the method for cryopreserving the cell, before the cell is frozen, moisture of the cell is removed.

Also, the method may include the steps of: preparing a composition for cryopreservation by mixing 5 to 50 vol % of serum, 5 to 10 vol % of ethylene glycol (EG), 5 to 10 vol % of propylene glycol, and 100 to 3000 ng/ml of antifreeze protein; and further suspending the cell by using the composition.

According to another aspect of the present invention, it provides a method for increasing a blastocyst development ratio of a cell, the method comprising the steps of: preparing a composition for cryopreservation by mixing 5 to 50 vol % of serum, 10 to 20 vol % of ethylene glycol (EG), 10 to 20 vol % of propylene glycol and 100 to 3000 ng/ml of antifreeze protein; suspending and cryopreserving the cell with the composition, and thawing the cell.

According to a further aspect of the present invention, it provides a composition for cryopreservation of a cell, obtained by adding an antifreeze protein at a concentration of 100 to 3000 ng/ml to a composition containing 5 to 50 vol % of serum, 5 to 20 vol % of ethylene glycol (EG), 5 to 20 vol % of propylene glycol and 40 to 55 vol % of buffer solution.

According to a still further aspect of the present invention, it provides a method for preparing a composition for cryopreservation of a cell, the method comprising the steps of: adding a serum to a buffer solution to prepare 5 to 50 vol % of serum solution; and adding 5 to 30 vol % of ethylene glycol (EG) and propylene glycol, and 100 to 3000 ng/ml of antifreeze protein to the serum solution so as to prepare the composition.

In the present invention, the antifreeze protein has a concentration ranging from 400 to 600 ng/ml, and is antifreeze protein type □.

In the present invention, the serum is selected from the group consisting of fetal bovine serum (FBS), bovine serum albumin (BSA) and synthetic substitute serum (SSS), and is preferably 20 vol % of fetal bovine serum.

In the present invention, the composition for cryopreservation further includes sucrose.

In the present invention, the term “cell” indicates a gamete (that is progenitors of a sperm or an oocyte), but the present invention is not limited thereto. However, for example, the cell may include a primordial germ cell, an embryonic germ cell, a testicular germ cell, a spermatogonial stem cell, and a germline stem cell. In the present invention, the cell may be selected from a group consisting of a human oocyte, a fertilized human egg, and an ovary tissue.

In the present invention, the term “antifreeze protein” indicates a protein having a function of preventing plants or animals from being frozen at a sub-zero temperature, and is generally discovered in the inside of the body of ocean fishes living in the polar region or from a plant inhabiting in a cold region. Also, the antifreeze protein includes, but is not limited to, for example, Type 1, 2, and 3.

The inventive cryopreservation method of an oocyte minimizes damage to the oocyte, which increases the survival rate of the oocyte after freezing/thawing of the oocyte, and improves a fertilization rate, and a blastocyst development ratio of the oocyte.

### BRIEF DESCRIPTION OF THE DRAWINGS

The foregoing and other objects, features and advantages of the present invention will become more apparent from the following detailed description when taken in conjunction with the accompanying drawings in which:

FIG. 1 is a graph showing the survival rate of an In-vivo matured MII oocyte according to the amount of an antifreeze protein; and

FIG. 2 is a graph showing the blastocyst development ratio of an In-vivo matured MII oocyte according to the amount of an antifreeze protein.

### DETAILED DESCRIPTION OF THE DISCLOSURE

Hereinafter, the present invention will be described in more detail with reference to Examples. However, Examples, as described below, are illustrative only, and do not limit the present invention.

### Example 1

### Oocyte Cryopreservation Through Addition of an Antifreeze Protein

An oocyte was suspended in 20 vol % fetal bovine serum (FBS) solution in HEPES-buffered TCM-199 (Gibco) as a basic medium for 5 minutes, and was separated. 20 ml of equilibrium solution (ES) containing 7.5 vol % ethylene glycol (1.5 ml), 7.5 vol % propylene glycol (1.5 ml) and basic medium 17 ml was prepared. The separated oocyte was suspended in 700 μl of equilibrium solution for 5 minutes. Then, the oocyte was separated again. A vitrification solution (VS) containing 15 vol % ethylene glycol (3 ml), 15 vol % propylene glycol (3 ml), 0.5M sucrose (3.424 g/20 ml) and basic medium 14 ml was prepared. The separated oocyte was suspended in 700 μl of vitrification solution for less than 1 minute. 5 to 6 oocytes on the vitrification solution (VS) were replaced on CryoTop (Kitazato, Japan), and their moisture was removed. Immediately, they were immersed within liquid nitrogen and stored in a nitrogen tank. Herein, each the equilibrium solution (ES) and the vitrification solution (VS) was added with AntiFreeze Protein type III at a concentration of 500 ng/ml. Then, the development ratio was observed as compared to a control group to which the AntiFreeze protein type III was not added. The oocytes were obtained from mice.

A cryopreservation composition added with an antifreeze protein at a concentration of 500 ng/ml was used, and then a survival rate, a fertilization rate, and a blastocyst development ratio of an In-vivo matured MII oocyte were measured. As a result, unlike the control group to which the antifreeze protein was not added, all of the survival rate, the fertilization rate, and the blastocyst development ratio were significantly increased. Accordingly, it can be found that the addition of the antifreeze protein can minimize the damage to a spindle fiber and a cell structure during cryopreservation of an oocyte. Thus, it is determined that the inventive cryopreservation liquid composition is effective (Table 1).

TABLE 1

| | | AFP | |
|---|---|---|---|
| | control | treatment | |
| | group (%) | (%) | P |
| Initiated in-vivo | 194 | 223 | |
| matured oocyte | | | |
| (Initiated MII) | | | |
| Surviving oocyte | 164 (84.54%) | 211 (94.62%) | 0.001 |
| (Surviving after | | | |
| vitrification-warming) | | | |
| Cleaved after | 132 (80.49%) | 200 (94.79%) | *<0.01 |
| insemination | | | |
| Blastocyst | 113 (85.61%) | 188 (94.00%) | 0.01 |
| development(Blastocyst, | | | |
| per cleaved) | | | |
| Blastocyst development | 113 (68.90%) | 188 (89.10%) | *<0.01 |
| (Blastocyst, per | | | |
| survived) | | | |

Also, the cryopreservation compostion added with the antifreeze protein at a concentration of 500 ng/ml was used to an immature oocyte, and then a survival rate, a fertilization rate, and a blastocyst development ratio of an immature oocyte were measured. As a result, the survival rate was significantly higher than that of an In-vivo matured MII oocyte. There was a statistically significant difference in the survival rate between the control group and the experimental group. In other words, it was determined that the experimental group is more effective than control group (control group: 91.67%, experimental group: 100%). Meanwhile, there was no difference in a maturity rate between two groups. From this result, it is assumed that after an immature oocyte was thawed, the culture condition was also good. After intrauterine insemination, a fertilization rate (cleavage) of an oocyte was significantly high in the control group (control group: 66.67%, experimental group: 95.65%). The reason for this result is assumed that at the initial freezing, an oocyte structure related to fertilization was well protected. In the blastocyst development ratio with respect to the ratio of fertilized oocyte, there is no difference between the two groups. However, in the blastocyst development ratio with respect to the ratio of initial immature oocyte, the experimental group showed a significantly high ratio (Table 2).

TABLE 2

| | Control | AFP treatment | |
|---|---|---|---|
| | group (%) | (%) | P |
| Vitrification groups | GV stage | GV stage | |
| | control | AFP 500 ng/ml | |
| Initiated CEOs | 60 | 60 | |
| Surviving CEOs | 55 (91.67%) | 60 (100%) | *0.002 |
| after vitrification | | | |
| Matured oocyte (IVM rate) | 45 (81.82%) | 46 (76.67%) | NS |
| Cleaved after insemination | 30 (66.67%) | 44 (95.65%) | *<0.01 |
| (per matured) | | | |
| Blastocyst development | 21 (70.00%), | 35 (79.55%), | NS |
| (BR per cleaved, per initiated | 21 (35.00%) | 35 (58.33%) | *0.01 |
| CEOs) | | | |

### Example 2

### Cryopreservation of Oocyte According to the Weight Ratio of an Antifreeze Protein

Antifreeze protein was added in different weight ratios of 100 ng/ml, 500 ng/ml, 1 μg/ml, 10 μg/ml and 30/ml, a survival rate, a fertilization rate, and a blastocyst development ratio of an In-vivo matured MII oocyte were measured according to the weight ratio of the antifreeze protein. This experiment was carried out in the same manner as described in Example 1 except that the weight ratios of the antifreeze protein were varied.

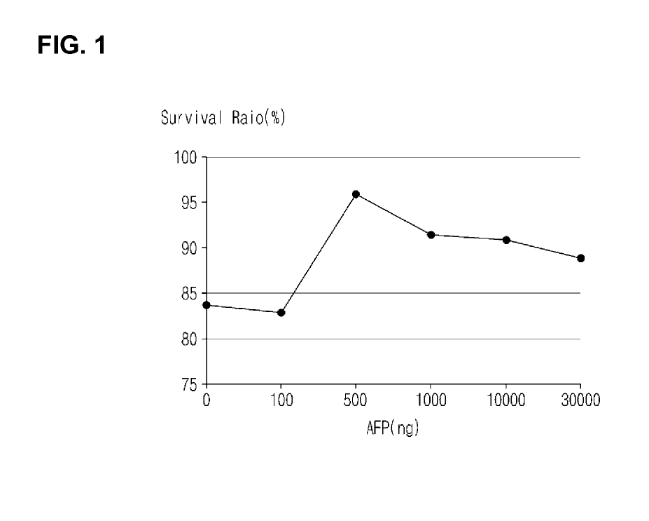

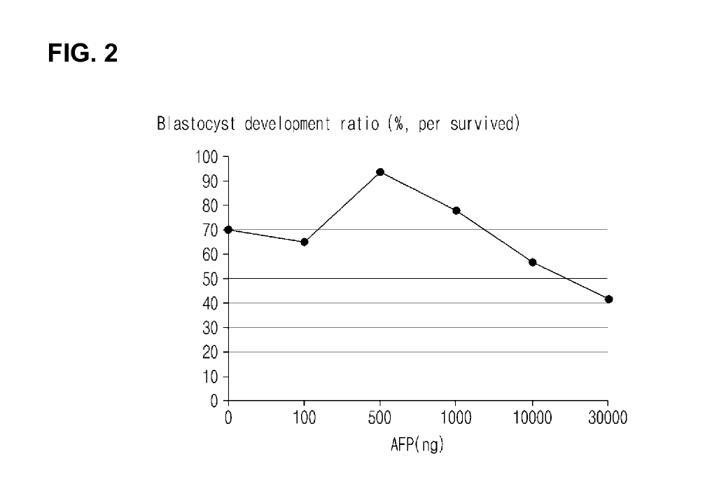

As a result, when the antifreeze protein was added at a weight ratio of 500 ng/ml, oocyte showed very significant good results in a survival rate, a maturity rate, a fertilization rate (cleavage rate), and a blastocyst development ratio (see FIGS. 1 and 2). From this, it was identified that a specific weight ratio of an added antifreeze protein is an important factor in determining the survival rate, the maturity rate, the fertilization rate, and the blastocyst development ratio of the oocyte in oocyte cryopreservation.

TABLE 3

| | Control | AFP | AFP | AFP | AFP | AFP |
|---|---|---|---|---|---|---|
| | group (%) | 100 ng/ml | 500 ng/ml | 1 μg/ml | 10 μg/ml | 30 μg/ml |
| Initiated matured | 43 | 41 | 49 | 35 | 33 | 27 |
| oocyte | | | | | | |
| (Initiated MII) | | | | | | |
| Surviving oocyte | 36 | 34 | 47 | 32 | 30 | 24 |
| (Surviving after | (83.72%) | (82.93%) | (95.92%) | (91.43%) | (90.91%) | (88.89%) |
| vitrification-warming) | | | | | | |
| Cleaved after | 29 | 28 | 46 | 28 | 21 | 16 |
| insemination | (80.56%) | (82.35%) | (97.87%) | (87.50%) | (70.00%) | (66.67%) |
| Blastocyst | 23 | 22 | 44 | 25 | 17 | 10 |
| development | (79.31%) | (78.57%) | (95.65%) | (89.29%) | (80.95%) | (62.50%) |
| (Blastocyst (per | | | | | | |
| cleaved)) | | | | | | |
| Blastocyst | 23 | 22 | 44 | 25 | 17 | 10 |
| development | (69.89%) | (64.71%) | (93.62%) | (78.13%) | (56.67%) | (41.67%) |
| (Blastocyst (per | | | | | | |
| survived)) | | | | | | |

### Example 3

### Formation of Spindle Fibers and Separation of Chromosomes According to Addition of an Antifreeze Protein

After an In-vivo matured oocyte was vitrified with addition of an antifreeze protein at a ratio of 500 ng/ml, the formation of spindle fibers and the separation of chromosomes were observed. As a result, it was found that the number of oocytes maintaining their normal status was increased (Table 4).

A normal status and an abnormal status of spindle fibers were discriminated as follows. In normal spindle fibers, a nucleus is lined up with respect to an equatorial plate, and the spindle fibers are symmetrically disposed in a spindle form with respect to the nucleus. In abnormal spindle fibers, a nucleus is lost or disposed in a loose order without being lined up, or some of spindle fiber are lost or tangled and lumped together. The normal status is graded as follows. Grade 1 of a normal status indicates that the form is partially similar or very similar to that before freezing of an oocyte. Grade 2 of a normal status indicates that the disposition of a nucleus and the symmetry of spindle fibers are relatively normal, but spindle fibers are fractionally lost or the disposition of the nucleus is not completely uniform. As a result, it was found that as compared to a control group, an experimental group showed a significantly improved normal status of spindle fibers. Further, it was found that the number of oocytes showing a normal status of grade 1 was significantly increased in the experimental group.

TABLE 4

| | control group | AFP 500 ng/ml | |
|---|---|---|---|
| | (%) | (%) | P |
| Treatment | In vivo-matured | In vivo-matured | |
| Number of | 102 | 108 | |
| oocytes | | | |
| total normal | 73 (71.67) | 92 (85.19) | 0.016* |
| status | | | |
| Normal status | 50 (49.02) | 77 (71.29) | 0.001* |
| (grade 1) | | | |
| Normal status | 23 (22.55) | 15 (13.89) | 0.103 |
| (grade 2) | | | |
| Abnormal status | 29 (28.43) | 16 (14.81) | 0.016* |

### Example 4

### Preservation of a Cell Membrane Through Addition of an Antifreeze Protein

After an In-vivo matured oocyte was vitrified with addition of an antifreeze protein at a ratio of 500 ng/ml, the preservation effect of a cell membrane was observed. As a result, it was found that as compared to a control group, in a case where the antifreeze protein was added, the number of oocytes having undamaged cell membranes was increased (Table 5).

A test for determining the preservation effect of a cell membrane by the addition of an antifreeze protein was carried out. As a result, a surviving cell was colored fluorescent green, and a dead oocyte or an oocyte having a damaged membrane was colored red. An oocyte that survived but, was damaged was colored both fluorescent green and fluorescent red. According to the result of this Example, an experimental group showed a significantly higher ratio of surviving oocytes without damage to a cell membrane, as compared to a control group. Meanwhile, the experimental group showed a statistically significantly low ratio of surviving oocytes with damage to a cell membrane. However, there was no statistical significance between two groups in view of morphologically normal oocytes which had died by the dyeing process.

TABLE 5

| | control group | AFP 500 ng/ml | |
|---|---|---|---|
| | (%) | (%) | P |
| Number of | 105 | 112 | |
| Oocytes | | | |
| surviving oocyte | 84 (80.00) | 102 (91.07) | p = 0.02* |
| (not damaged) | | | |
| surviving oocyte | 9 (8.57) | 2 (1.79) | p = 0.023* |
| (damaged) | | | |
| dead oocyte | 12 (11.43) | 8 (7.14) | p = 0.275 |

Although several exemplary embodiments of the present invention have been described for illustrative purposes, those skilled in the art will appreciate that various modifications, additions and substitutions are possible, without departing from the scope and spirit of the invention as disclosed in the accompanying claims.

1-28. (canceled)

29. A method for cryopreserving a cell, the method comprising the steps of:
preparing a composition containing 5 to 50 vol % of serum, 10 to 20 vol % of ethylene glycol (EG) and 10 to 20 vol % of propylene glycol by mixing;
adding an antifreeze protein at a concentration of 100 to 3000 ng/ml to the composition; and
suspending and freezing cells selected from the group consisting of a human oocyte, a fertilized human egg, and an ovary tissue with the composition.

30. The method as claimed in claim 29, wherein moisture of the cell is removed and then the cell is frozen.

31. The method as claimed in claim 29, further comprising the steps of:
preparing a composition of 5 to 50 vol % of serum, 5 to 10 vol % of ethylene glycol (EG) and 5 to 10 vol % of propylene glycol by mixing;
adding an antifreeze protein at a concentration of 100 to 3000 ng/ml to the composition; and
suspending the cell selected from the group consisting of a human oocyte, a fertilized human egg, and an ovary tissue with the composition.

32. The method as claimed in claim 29, wherein the antifreeze protein is added at a concentration ranging from 400 to 600 ng/ml.

33. The method as claimed in claim 29, wherein the antifreeze protein is antifreeze protein type III.

34. The method as claimed in claim 29, wherein the serum is any one selected from the group consisting of fetal bovine serum (FBS), bovine serum albumin (BSA), and synthetic substitute serum (SSS).

35. The method as claimed in claim 29, wherein the serum is 20 vol % of fetal bovine serum (FBS).

36. The method as claimed in claim 29, wherein 15 vol % of ethylene glycol (EG) and 15 vol % of propylene glycol were added.

37. The method as claimed in claim 31, wherein 7.5 vol % of ethylene glycol (EG) and 7.5 vol % of propylene glycol were added.

38. The method as claimed in claim 29, wherein the composition for the cryopreservation further comprises sucrose.

39. The method as claimed in claim 29, wherein the cell is selected from the group consisting of a human oocyte, a fertilized human egg, and an ovary tissue.

40. A method for increasing a blastocyst development ratio of a cell, the method comprising the steps of:
preparing a composition for cryopreservation containing 10 to 50 vol % of serum, 10 to 20 vol % of ethylene glycol (EG) and 10 to 20 vol % of propylene glycol by mixing;
adding an antifreeze protein at a concentration of 100 to 3000 ng/ml thereto; and
suspending and cryopreserving the cell selected from the group consisting of a human oocyte, a fertilized human egg and an ovary tissue with the composition, and thawing the cell.

41. A composition for cryopreservation of a cell, obtained by adding an antifreeze protein at a concentration of 100 to 3000 ng/ml to a composition containing 5 to 50 vol % of serum, 5 to 20 vol % of ethylene glycol (EG), 5 to 20 vol % of propylene glycol and 40 to 55 vol % of buffer solution.

42. The composition as claimed in claim 41, wherein the antifreeze protein is at a concentration ranging from 400 to 600 ng/ml.

43. The composition as claimed in claim 41, wherein the antifreeze protein is antifreeze protein type III.

44. The composition as claimed in claim 41, wherein the serum is any one selected from the group consisting of fetal bovine serum (FBS), bovine serum albumin (BSA), and synthetic substitute serum (SSS).

45. The composition as claimed in claim 41, wherein the serum is 20 vol % of fetal bovine serum (FBS).

46. The composition as claimed in claim 41, wherein the ethylene glycol (EG) and propylene glycol are 15 vol % of ethylene glycol (EG) and 15 vol % of propylene glycol.

47. The composition as claimed in claim 41, wherein the buffer solution further comprises sucrose.

48. The composition as claimed in claim 41, wherein the cell is anyone selected from the group consisting of a human oocyte, a fertilized human egg and an ovary tissue.

49. A method for preparing a composition for cryopreservation of a cell, the method comprising the step of:
preparing 5 to 50 vol % of serum solution by adding serum to buffer solution;
adding 5 to 30 vol % of ethylene glycol (EG) and propylene glycol to above (or the) serum solution; and
adding an antifreeze protein at a concentration of 100 to 3000 ng/ml thereto,

50. The method as claimed in claim 49, wherein the antifreeze protein has a concentration ranging from 400 to 600 ng/ml.

51. The method as claimed in claim 49, wherein the antifreeze protein is antifreeze protein type III.

52. The method as claimed in claim 49, wherein the serum is any one selected from the group consisting of fetal bovine serum (FBS), bovine serum albumin (BSA), and synthetic substitute serum (SSS).

53. The method as claimed in claim 49, wherein the serum is 20 vol % of fetal bovine serum (FBS).

54. The method as claimed in claim 49, wherein the ethylene glycol (EG) and propylene glycol are 15 vol % of ethylene glycol (EG) and propylene glycol.

55. The method as claimed in claim 49, wherein the composition for the cryopreservation further comprises sucrose.

56. The method as claimed in claim 21, wherein the cell is anyone selected from the group consisting of a human oocyte, a fertilized human egg and an ovary tissue.
